(19) United States
(12) Patent Application Publication
(10) Pub. No.: US 2026/0083509 A1
(43) Pub. Date: Mar. 26, 2026

(54) MODELING TOOLS FOR TOTAL SHOULDER ARTHROPLASTY PRE-OPERATIVE PLANNING

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Alessandro Navacchia, Broomfield, CO (US); Samantha Wood, Cordova, TN (US); Nathan A. Netravali, Littleton, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/406,226

(22) Filed: Dec. 2, 2025

Related U.S. Application Data
(63) Continuation-in-part of application No. 19/103,914.
(63) Continuation-in-part of application No. PCT/US2024/048517, filed on Sep. 26, 2024.
(60) Provisional application No. 63/405,223, filed on Sep. 9, 2022.
(60) Provisional application No. 63/545,667, filed on Oct. 25, 2023.

(51) Int. Cl. A61B 34/10; A61F 2/46

(52) U.S. Cl. CPC A61B 34/10; A61F 2/4612; A61B 2034/104; A61B 2034/105; A61B 2034/108; A61F 2002/4633

(57) ABSTRACT

Disclosed herein is a system and method for performing pre-operative planning of total joint arthroplasty. The planning tool builds a model and analyzes and visualizes movement of the joint for various selections of implant models and placement of the components on the implants on the patient's anatomy. The tool also analyzes and visualizes motions of the joint during common activities of daily living and analyzes and functional ranges. The tool provides real time clinically relevant parameters to inform pre-surgery planning. The tool provides screw orientation visualization.

### RELATED APPLICATIONS

This application is a continuation-in-part of U.S. patent application Ser. No. 19/103,914, filed Feb. 14, 2025, which is a National Phase filing of International Application No. PCT/US2023/032212, filed Sep. 7, 2023, which application claims the benefit of U.S. Provisional Patent Application Ser. No. 63/405,223, entitled “Modeling Tools for Total Shoulder Arthroplasty Pre-Operative Planning” filed Sep. 9, 2022.

This application is a continuation-in-part of International Application No. PCT/US2024/048517, filed Sep. 26, 2024, which application claims the benefit of U.S. Provisional Patent Application No. 63/545,667, filed Oct. 25, 2023, entitled “System and Method for Determining Personalized Placement and Selection of Shoulder Implants.”

The contents of each application is incorporated herein in its entirety.

### FIELD

The present disclosure relates generally to orthopedic procedures, for example, total joint replacement procedures, and, in particular, to total shoulder arthroplasty (TSA) procedures.

### BACKGROUND

Orthopedic procedures to replace joints, for example, shoulders, are well known and commonplace in today's society. Total shoulder replacement, also known as total shoulder arthroplasty (TSA), is the removal of portions of the shoulder joint, which are replaced with artificial implants to reduce pain and restore range of rotation and mobility. In an anatomic TSA procedure, which may be used, for example, when osteoarthritis has compromised portions of the shoulder joint, the ball portion of the shoulder joint is attached to the top of the humerus and the cup portion of the shoulder joint is attached to the glenoid. In a reverse TSA procedure, which may be used, for example, when the rotator cuff has been compromised, the ball portion of the shoulder joint is placed on the glenoid while the cup portion of the shoulder joint is attached to the top of the humerus.

Current state-of-the-art planning for TSA consists of using a computed tomography (CT) scan from the patient to correctly size and position implant components on the patient's specific anatomy. One of the objectives of TSA is to restore normal shoulder function, which includes both mobility and stability. Pre-operative prediction of the implanted joint range-of-motion (ROM) can facilitate optimization of post-operative mobility. Collision between the humeral liner and the scapula due to limited ROM in adduction has been identified as a cause of scapular notching, which has been reported to occur in more than half of reverse TSA patients and has been related to glenoid loosening and implant failure. In addition, limited ROM and impingement can lead to implant instability and dislocation, which is one of the leading causes for revision surgery. Reverse TSA jump distance calculation can provide insight into the trade-off between mobility and stability of the joint.

In addition, post-operative mobility and stability of the joint are significantly impacted by muscle function. Although this is true for every joint replacement, this is especially relevant to TSA as joint stability is primarily provided by muscle compressive forces and implant design (e.g., anatomic vs reverse) has a significant impact on muscle moment arms and muscle force generation capacity. Therefore, pre-operative knowledge of patient-specific post-operative muscle function can further improve the surgeon's ability to optimally select implant design, size, and placement.

Muscle forces are the main determinant of contact mechanics. Large rotational moments at the implant-bone interface due to the joint contact force line of action can lead to glenoid component loosening (e.g., “rocking-horse” failure mechanism for anatomic TSA). Therefore, prediction of contact mechanics during activities of daily living can provide valuable insight into the risk of failure of the implant.

Several commercially-available planning applications provide the ability to calculate shoulder impingement-free ROM in its primary degrees of freedom (i.e., flexion/extension, abduction/adduction, internal/external rotation) by rotating the implanted humerus about the three axes of rotation and detecting at what rotation bone-on-bone or implant-on-bone impingements occur. However, joint ROM in its main degrees of freedom is hard to link to the mobility needs of a patient during daily life. Therefore, although TSA is supposed to restore joint mobility, the knowledge of the specific mobility patterns required by a patient to perform activities of daily living would better inform surgery planning.

Generic knowledge of how implant design (anatomic vs reverse) affects post-operative muscle function is available in the scientific literature. However, it is difficult to extrapolate such generic knowledge to a specific patient, given the broad spectrum of osteoarthritic joint deformity and pre-operative muscle state. Currently available planning tools do not incorporate any prediction of post-operative muscle function nor do they predict the post-operative ROM or the ability of the patient to perform activities of daily living post-operatively.

### SUMMARY

Disclosed herein is a system and method for providing surgical pre-planning of both anatomic and reverse TSA procedures. 3D modeling tools are used together with CT-based patient anatomy within TSA planning software to simulate post-operative shoulder ROM in the joint's primary degrees of freedom to provide insight into the TSA mobility-stability trade-off on a patient-specific basis. Additionally, the present disclosure provides simulation of post-operative glenohumeral motion during activities of daily living relevant to normal shoulder function to inform implant selection and placement with real life mobility patterns. The present disclosure also simulates post-operative muscle (i.e., deltoid, rotator cuff) function (i.e., moment arm, length, force generating capacity) to inform implant selection and placement. Lastly, the present disclosure performs muscle-driven simulations of activities of daily living to predict implant contact mechanics and estimate risk of glenoid component loosening.

In a first example, a method for performing pre-operative planning of joint replacement surgery comprises deriving a model of the anatomy of a joint to be replaced from images gathered from one or more imaging modalities, receiving a selection of an implant and placement of the implant on the anatomy of the joint and analyzing and visualizing, on the model of the joint, a range-of-motion of the joint for the selected implant and placement of the implant.

In any preceding or subsequent example, the method further comprises visualizing the placement of the implant on the model of the joint.

In any preceding or subsequent example, the method further comprises providing an animated visualization of the model through a range-of-motion for each major axis of rotation.

In any preceding or subsequent example, the method further comprises wherein the animated visualization automatically moves the model through the ranges-of-motion.

In any preceding or subsequent example, the method further comprises wherein the animated visualization is controlled using a manual input of the ranges-of-motion.

In any preceding or subsequent example, the method further comprises providing a graphical visualization of the range-of-motion with respect to each of the three axes of motion.

In any preceding or subsequent example, the method further comprises wherein the graphic visualization of the ranges-of-motion is a graph.

In any preceding or subsequent example, the method further comprises wherein the graphic visualization of the ranges-of-motion is an animated skeletal representation of the joint.

In any preceding or subsequent example, the method further comprises providing one or more views of the imagery from which the model was derived showing placement of components of the implant thereon.

In any preceding or subsequent example, the method further comprises providing an animated visualization of the ranges-of-motion of the model required for performance of one or more activities of daily living.

In any preceding or subsequent example, the method further comprises informing the user of parts of the bone and/or implant that are impinging or colliding during range-of-motion visualizations.

In any preceding or subsequent example, the method further comprises highlighting parts of the patient's anatomy where impingement occurs.

In any preceding or subsequent example, the method further comprises providing a graphical visualization of the ranges-of-motion with respect to each of the three axes of rotation for the one or more activities of daily living.

In any preceding or subsequent example, the method further comprises providing a selection mechanism for selecting the activity of daily living to be animated.

In any preceding or subsequent example, the method further comprises providing a visualization of the change in a location of a center of rotation of the joint on a visualization of the model.

In any preceding or subsequent example, the method further comprises wherein the visualization of the model comprises one or more muscles visualized on the model.

In any preceding or subsequent example, the method further comprises providing a selection mechanism for selecting the muscles to be visualized on the model.

In any preceding or subsequent example, the method further comprises providing a graphical visualization of the moment arm of the one or more muscles selected with the selection mechanism.

In any preceding or subsequent example, the method further comprises providing a graphical visualization of the force-length curve through relevant ranges-of-motion for the one or more muscles selected with the selection mechanism.

In any preceding or subsequent example, the method further comprises receiving a selection of one or more additional implant models and placement of the models on the patient's anatomy.

In any preceding or subsequent example, the method further comprises providing a comparison between the originally-selected implant model and the one or more additional selected implant models.

In any preceding or subsequent example, the method further comprises providing a graphical visualization of the range-of-motion with respect to each of the three axes of rotation for each of the selected implant models.

In any preceding or subsequent example, the method further comprises suggesting implant positions that optimize certain parameters such as muscle moment arm, force-length curve, or minimal impingement.

In another example, a system for performing pre-operative planning of joint replacement surgery comprises a processor and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, providing an application for analyzing and visualizing on the model a range-of-motion of the joint for a particular implant and placement of components of the implant on the anatomy of the joint.

In any preceding or subsequent example, the software causes the system to perform the further function of providing an application for analyzing and visualizing a range-of-motion of the joint required for performance of one or more activities of daily living.

In any preceding or subsequent example, the software causes the system to perform the further function of providing application for analyzing and visualizing a change in a location of a center of rotation of the joint in a visualization of the model, wherein the visualization of the model comprises one or more muscles displayed on the model.

In any preceding or subsequent example, the system further comprises wherein the processor is a surgical computer provided as part of a computer-assisted surgical system.

In another example, a system for performing pre-operative planning of joint replacement surgery comprises a processor and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, providing an application for analyzing and visualizing on the model, a location of a center of rotation of the joint in a visualization of the model, wherein the visualization of the model comprises one or more muscles displayed on the model.

In another example, a method of identifying impingement points in pre-operative planning of joint replacement surgery includes deriving a model of the anatomy of a joint to be replaced from images gathered from one or more imaging modalities, receiving a selection of an implant and placement of the implant on the anatomy of the joint, analyzing and visualizing, on the model of the joint, a range-of-motion of the joint for the selected implant and placement of the implant, and providing an animated visualization of the model through the range-of-motion, wherein the animation applies scapulothoracic and glenohumeral angles to the model to simulate one or more of activities of daily living and functional ranges.

In any preceding or subsequent example, the method further comprises identifying, in the animation, one or more instances in which an impingement of the joint or the implant is indicated in the animation.

In any preceding or subsequent example, the method further comprises wherein the impingement is indicated via a visual interface object.

In any preceding or subsequent example, the method further comprises wherein the visual interface object changes a display based on a number of simulation frames that indicate impingements.

In any preceding or subsequent example, the method further comprises wherein the indicating of the impingement occurs in real time to provide data for selection of a placement of the implant.

In any preceding or subsequent example, the method further comprises wherein the animation of the ranges of motion comprises an animated skeletal representation of the joint.

In another example, a method for performing pre-operative planning of joint replacement surgery comprises deriving a model of the anatomy of a joint to be replaced from images gathered from one or more imaging modalities, based on the model, calculating one or more of Acromiohumeral Distance (AHD), Lateral Humeral Offset (LHO), Rotation Center Change, and a difference between the glenosphere center to acromion distance (DA) and the glenosphere center to greater tuberosity distance (DGT), and displaying the calculations in real time on a user interface while receiving a selection of an implant and placement of an implant on the anatomy of the joint.

In any preceding or subsequent example, the method further comprises wherein the calculations are based on a scapula coordinate system that is defined using three landmarks: a glenoid center, a medial border, and an inferior angle.

In any preceding or subsequent example, the method further comprises wherein the AHD is measured as a vertical distance between an acromion most lateral point and a humerus greater tuberosity highest point when both a scapula and humerus are aligned to a neutral position of the joint.

In any preceding or subsequent example, the method further comprises wherein the LHO is measured as a horizontal distance between an acromion most lateral point and a humerus most lateral point when both a scapula and the humerus are aligned to their neutral position.

In any preceding or subsequent example, the method further comprises wherein the rotation center change is measured as a distance between a native humerus head center and an implant rotation center when both a scapula and a humerus are aligned to neutral position.

In any preceding or subsequent example, the method further comprises wherein the rotation center change is divided into three components of superior-inferior, medial-lateral, and anterior-posterior.

In any preceding or subsequent example, the method further comprises wherein the neutral position is identified by squaring the scapula and humerus coordinate systems to a global coordinate system.

In another example, a method of planning screw placements during pre-operative planning of joint replacement surgery comprises deriving a three-dimensional model of the anatomy of a joint to be replaced from images gathered from one or more imaging modalities, receiving a selection of an implant and placement of the implant on the anatomy of the joint, selecting a two-dimensional cross section of the three-dimensional model, and rotating a selected screw through the two-dimensional cross section to identify a placement.

In any preceding or subsequent example, the method further comprises wherein the identified placement indicates that the screw placement causes a bi-cortical fixation.

In any preceding or subsequent example, the method further comprises wherein the screw is rotatable along a 20-degree cone of rotation.

In any preceding or subsequent example, the method further comprises wherein a user interface of the model provides an indication when a placement of the screw indicates a bi-cortical fixation.

In any preceding or subsequent example, the method further comprises wherein the screw is selectable via an interface object on a user interface of the model.

In another example, a system is provided for performing pre-operative planning of joint replacement surgery comprising a processor and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, and providing an application for analyzing and visualizing on the model a range-of-motion of the joint for a particular implant, and providing an animated visualization of the model through the range-of-motion, wherein the animation applies scapulothoracic and glenohumeral angles to the model to simulate one or more of activities of daily living and functional ranges.

In another example, a system is provided for performing pre-operative planning of joint replacement surgery comprising a processor, and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, based on the model, calculating or more of Acromiohumeral Distance (AHD), Lateral Humeral Offset (LHO), Rotation Center Change, and a difference between a glenosphere center to acromion distance (DA) and the glenosphere center to a greater tuberosity distance (DGT), and displaying these calculations in real time on a user interface while receiving a selection of an implant and placement of an implant on the anatomy of the joint.

In another example, a system is provided for performing pre-operative planning of joint replacement surgery comprising a processor, and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, receiving a selection of an implant and placement of the implant on the anatomy of the joint, selecting a two-dimensional cross section of the three-dimensional model, and rotating a selected screw through the two-dimensional cross section to identify a placement that achieves bi-cortical fixation.

Examples of the present disclosure provide numerous advantages. For example, pre-operative planning performance is improved via the enhanced prediction of post-operative mobility and stability. Additionally, the disclosed system and method provide more personalized planning which can lead to clinical benefits such as improved patient function and reduced risk of dislocation or failure. The disclosed system and method further enhance the ability of the surgeon to interpret several patient-specific biomechanical quantities.

Further features and advantages of at least some of the examples disclosed herein, as well as the structure and operation of various examples, are described in detail below with reference to the accompanying drawings.

### BRIEF DESCRIPTION OF THE DRAWINGS

By way of example, specific examples of the disclosed system and method will now be described, with reference to the accompanying drawings, in which:

FIG. 1 is a block diagram showing an exemplary workflow in accordance with one or more features of the present disclosure.

FIG. 2 shows an exemplary user interface screen from the range-of-motion analysis tool.

FIG. 3 shows an exemplary user interface screen from the activities of daily living analysis tool.

FIG. 4 shows an exemplary user interface screen from the muscle analysis tool.

FIG. 5 shows an exemplary user interface screen from the comparison tool.

FIG. 6 depicts an operating theatre including an illustrative computer-assisted surgical system with which various examples of the disclosed system and method could be used.

FIG. 7 depicts native or acquired posture types.

FIG. 8A depicts a scapulothoracic orientation angle.

FIG. 8B depicts an arm elevation comprised of both a glenohumeral elevation and a scapulothoracic elevation.

FIG. 9 depicts a functional position of a shoulder.

FIG. 10 depicts an acromiohumeral distance and a lateral humeral offset.

FIG. 11 depicts a rotational center change.

FIG. 12 depicts a screw planning user interface for the system.

### DEFINITIONS

For the purposes of this disclosure, the term “implant” is used to refer to a permanent or temporary prosthetic device or structure manufactured to replace or enhance a biological structure.

Although much of this disclosure refers to surgeons or other medical professionals by specific job title or role, nothing in this disclosure is intended to be limited to a specific job title or function. As such, the term “user”, as used herein should be interpreted to include any doctor, nurse, medical professional, or technician, or any other user of the system or practitioner of the method. Any of these terms or job titles can be used interchangeably with the user of the systems disclosed herein unless otherwise explicitly stated.

### DETAILED DESCRIPTION

The system and method disclosed herein provides a tool for pre-operative planning of a joint replacement surgery which will assist the user in selecting the proper implant and planning and executing the surgery such as to minimize both intra-operative and post-operative negative outcomes for the patient. Note that, although the disclosed system and method are explained in terms of a total shoulder replacement procedure, all or portions of the disclosed system and method may be modified such as to be applicable to any joint replacement procedure.

In a TSA procedure, the different approaches to the surgery provide different outcomes. For example, in an anatomic TSA procedure, the result provides in most cases similar range-of-motion and similar biomechanics to the user's native anatomy. A reverse TSA procedure, on the other hand, tends to provide reduced range-of-motion and biomechanics that have been altered from the user's native anatomy. For example, in the reverse procedure, the range-of-motion may be limited by bone-to-bone, implant-to-implant, or implant-to-bone impingement, which may lead to instability of the joint. Additionally, the reverse procedure may result in a medialized center of rotation and humerus distalization, which results in changes in the length, moment arms and function of the various muscles of the joint.

The present disclosure provides the user with a means for predicting and evaluating post-operative results given the variables of the type of procedure to be used, the specific implant selected, the particular placement of the implant on the patient's anatomy and the changes to the patient's anatomy resulting from the selection and placement of a particular implant, both for the anatomical procedure and the reverse procedure. The present disclosure provides a tool for analyzing and visualizing a predicted range-of-motion (RoM), given various selections of implants and placements thereof, a tool for analyzing and visualizing a predicted ability of the patient to perform various activities of daily living and a tool for analyzing and visualizing predicted changes to the patient's anatomy, namely, the changes to the center of rotation of the joint and the moment arm and length of the muscles. In certain examples, the system and method may provide recommendations for optimized outcomes based on preferences of the surgeon and/or patient.

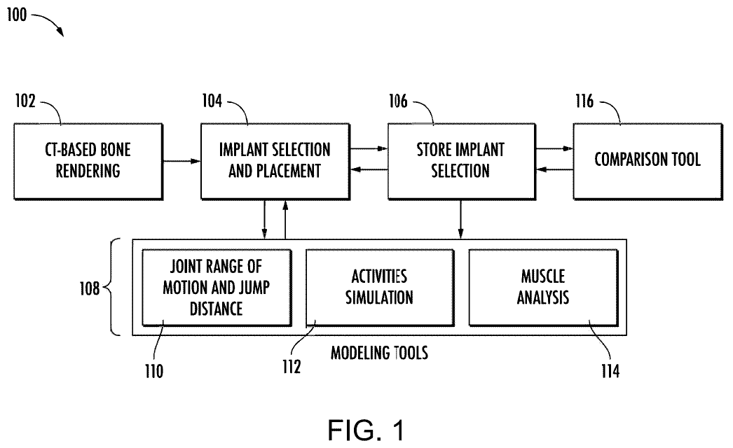

FIG. 1 is a block diagram showing an exemplary workflow 100 in accordance with one or more features of the present disclosure. The present disclosure relies on the creation of a model of the anatomy of the specific patient showing the bone structures that will be affected by placement of the implant. In certain examples, at step 102 of workflow 100, the model may be derived from a CT scan. In other examples, the model may be derived using one or more other imaging modalities, including, for example, MRI, X-ray or ultrasound, or various combinations thereof. For example, a CT scan may show the bones of the joint while an MRI can show soft tissue structures in addition to bones. In certain examples, the model can also be derived using an atlas or statistical shape model based on information gathered intraoperatively using anatomical landmarks gathered during procedures.

At step 104 of workflow 100, the surgeon specifies to the tool the type of surgery to be performed, a selection of a particular model of implant (including various characteristics of the implant, for example, size of the various components) and a specific placement of the implant on the patient's anatomy. In certain examples, workflow 100 can also suggest an initial placement of the implant based on the anatomy of the patient or on a simulation.

At step 106 of workflow 100, the specific implant selection and placement from step 102 will be stored for use by the analysis tools. At this point in workflow 100, the user can access the suite 108 of analysis and visualization tools which include, in some examples, a tool providing a range-of-motion analysis 110, a tool for simulating activities of daily living 112 and a muscle analysis tool 114, based on the model of the patient's anatomy and the specific implant selection and placement information provided in step 104 of workflow 100. The analysis tools in suite 108 will be explained in more detail below. At any time during the use of the analysis and visualization tools in suite 108, the user may change the implant selection and placement information.

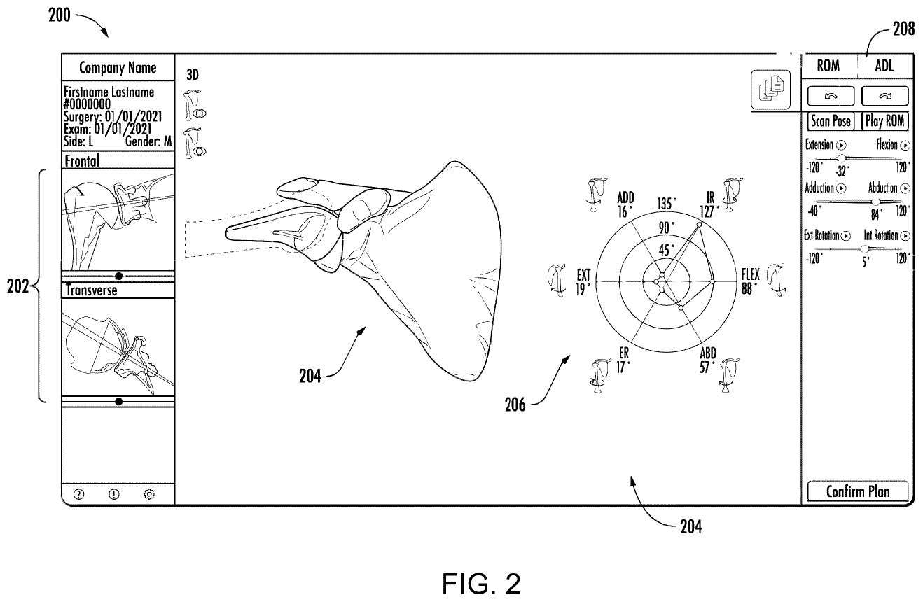

An exemplary screen 200 of the range-of-motion analysis and visualization tool 110 is shown in FIG. 2. As would be realized by one of skill in the art, the arrangement of information on the screen and the manner of expressing the information may be changed without departing from the spirit or scope of the present disclosure.

In one example, user interface 200 shows one or more views 202 from the imagery used to generate the model of the patient's bone anatomy. The one or more views 202 may show the placement of the various components of the implant overlaid on the imagery. In the specific case shown in FIG. 2, the implant components for a reverse TSA procedure are shown.

Exemplary user interface 200 may also show a model 204 of the joint which has been derived from the imagery collected in step 102 of workflow 100. The model 204 shows the specified positioning of the components of the selected implant. In certain examples, the model may be animated to show a kinematic simulation of glenohumeral motion. The model of the joint 204 may be placed in various positions using manual controls shown in area 208 of user interface 200. In some examples, the manual control may be sliders, as shown in FIG. 2. Alternatively, or in addition to, the tool may automatically move the model of the joint 204 through a full range of possible motions, which are visualized on an animation of model 204.

The method calculates impingement-free ranges-of-motion in the shoulder's main degrees of freedom (i.e., abduction/abduction, flexion/extension, interior rotation/exterior rotation) for the current implant selection and placement chosen by the user. Each time the user changes the implant or placement, the software calculates the joint ranges-of-motion by sequentially rotating the bone and implant geometries about the three main axis of rotation and detects bone-on-bone, implant-on-implant, and/or implant-on-bone impingement using a collision detection algorithm. In some examples, the user may be informed of the parts of the patient's anatomy wherein impingement which limits the range-of-motion occurs, for example, by highlights those parts of the anatomy.

A visualization 206 of the joint ranges-of-motion in the primary degrees of freedom is presented, with the polygon denoting the available range-of-motion without impingement. As would be realized by one of skill in the art, many other methods of denoting the available range-of-motion without impingement could be used. An additional indicator of the available range-of-motion is shown in area 208 of user interface 200 which shows the colored portions of the sliders to indicate the available range-of-motion while the gray areas of the sliders indicate areas of impingement. The slider bars in area 208 of user interface 200 may also be used to manually control the joint angles shown in animated model 204.

For reverse TSA procedures, calculation of and a visualization of the jump distance may be presented on this or on a different screen (not shown). The jump distance is the distance that the glenosphere needs to travel to dislocate from the liner, after impingement in the primary degrees of freedom. The jump distance is a quantification of joint conformity and stability provided purely by the geometry of the components. Because implant performance is a trade-off between mobility and stability, it is important to evaluate both the ranges-of-motion and the jump distance simultaneously when selecting implant parameters and placement.

The user may change the selection of or placement of the components of the implant at any time. This may be accomplished by returning to step 104 or workflow 100. Within user interface 200, this may be accomplished by clicking on the one or more views 202 of the native imagery. Alternatively, controls (not shown) may be provided directly on screen 202 to affect a change in the selection or placement of the implant.

The modeling tools may be used to further detect impingement potential and risk based on the bone anatomy of the patient, the simulated model, and placement of the implant. In the model, the joint movement of the scapula and the humerus in a simulation that is based on the patient anatomy allows for the model to identify potential impingement points that may be created when both the scapula and the humerus are in motion.

The scapula protraction angle (also referred to as an internal rotation angle) is measured as an angle between the scapula Friedman's line (medio-lateral axis) and the CT volume horizontal axis.

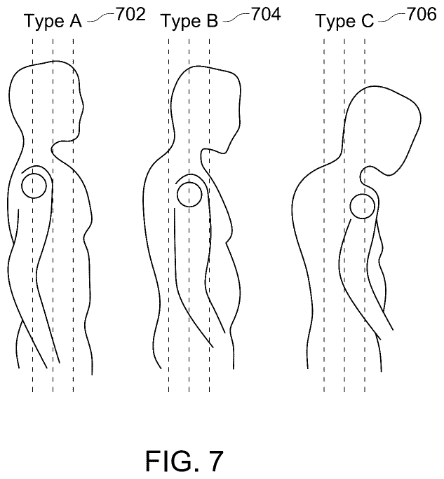

To more accurately define the scapula protraction angle and other aspects of the model, native or acquired posture types are illustrated in FIG. 7. In the illustration, the posture of the patient is classified as Type A 702, Type B 704, or Type C 706 based on the measure of the internal rotation of the scapula. Postures differ between people and, in particular, subjects with a more kyphotic posture present larger scapula protraction and internal rotation in a neutral position. In turn, scapula posture affects the position of the humerus with respect to the glenoid in the neutral position. Patient posture can be type classified pre-operatively by measuring the scapula internal rotation angle with respect to the torso, as illustrated in FIG. 1, which shows three different native or acquired posture types (A-C), with progressive scapular protraction, tilt, and internal rotation, as well as drooping. CT-based scapula reconstruction can be used to assign patients to each group type A, B, or C. As illustrated, the patient's scapular protraction, tilt, and internal rotation progress with each type.

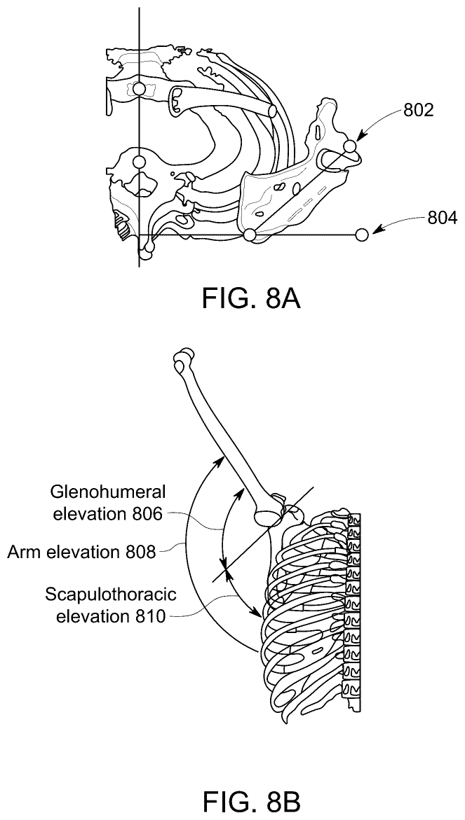

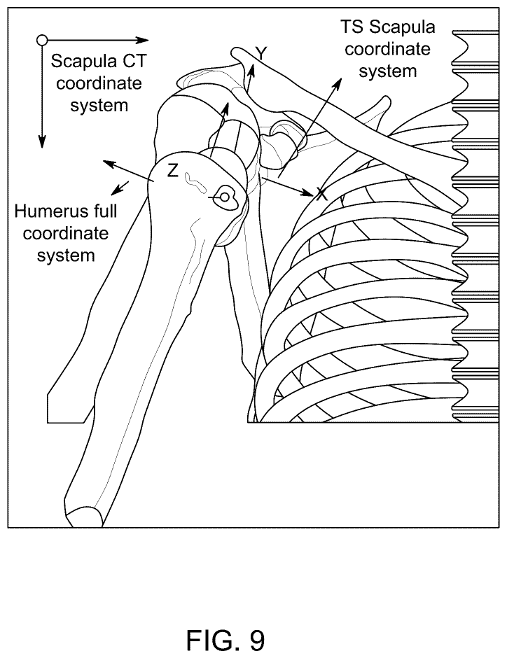

FIG. 8A illustrates a scapulothoracic orientation angle that is measured in the different postures of FIG. 7. Scapulothoracic orientation is defined as the angle between the scapula plane 802 and the coronal plane 804 of the body.

In the illustrations of FIG. 7, a Type A 702 has in internal rotation≤36°; a Type B 704 has an internal rotation in the range of 36° to 46°; and a Type C 706 has an internal rotation≥47°. In various examples, these measures may differ slightly based on the user populations, measurement techniques or other factors. Simulation and clinical evaluations demonstrated that scapulothoracic orientation has an impact on theoretical range of motion (ROM) and ROM clinical outcomes.

FIG. 8B illustrates an arm elevation 808 comprised of both a glenohumeral elevation 806 and a scapulothoracic elevation 810.

In an arm elevation 808, the humerus is raised via a combination of glenohumeral elevation 806 and scapulothoracic elevation 810. By applying scapulothoracic and glenohumeral angles to the implanted virtual model, the system is able to simulate activities of daily living (ADL). The ADL may include activities such as washing axilla, perineal care, fastening a seatbelt, combing one's hair, attaching a bra, putting on a belt, and other related activities.

The system may also simulate Functional Ranges. These functional ranges may include motions such as arm elevation, external rotation, reaching opposite, reaching behind head, reaching back, maximum reach back, and other motions. Joint angles for the different simulations may be derived from a skeletal model of the body driven by motion capture marker data.

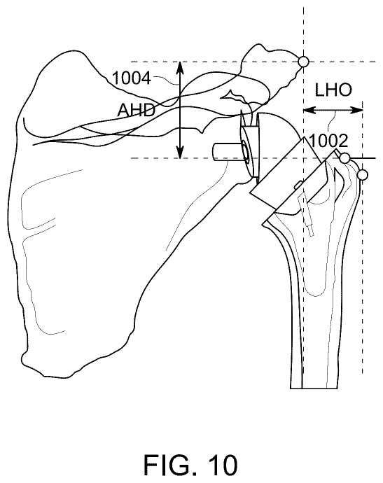

FIG. 9 illustrates a functional position of a shoulder.

ADL and Functional Ranges impingement calculations may employ a particular neutral shoulder position, called Functional Position. To position bones and implants in the Functional Position, the model of the shoulder is manipulated into a particular arrangement.

First, the system loads the scapula CT coordinate system using a configured transformation software option when loading the case. The model is configured as a “child” of the scapula. Next, the system moves the scapula to align the scapula CT coordinate system to the global axes. This position is illustrated in FIG. 9. In an example, the system then sets the humerus (and implants attached to it) orientation to have a configured set of Euler angles (x-z-y sequence) between the humerus full CS and the scapula TS CS. In an example, the Euler angles may be configured as:
right side: (−18.2, 3.0, −38.6)
left side: (−18.2, −3.0, 180.0+38.6)

The system then translates the humerus (and attached implants) to assemble the implant joint.

For ADLs, the system is configured to detect simulation frames that indicate impingement may be occurring. The system communicates the amount of impingement to the user via the user interface of the software or in any other suitable manner.

In the software, each ADL included in the planner will have an icon that can be selected that will initiate a corresponding animation to visualize the ADL. In addition to the initiation of the animation, each icon is surrounded by a circle or other visual indication that communicates to the user if impingement occurs during the ADL motion. The communication may be indicated using a color change or other visual indication of an impingement. For example, if the circle is a green full circle, the ADL does not present impingement, whereas impingement is present if the circle is not fully green and/or includes red portions. The impingement indication may be scaled to indicate the extent or duration of the impingement. For example, the red portion of the circle may indicate how many simulation frames have impingement relative to the total number of frames for that ADL. During planning, the ADL icons update in real time as soon as the user makes any changes to the implant placement and selection.

The software may include an animation progress bar. When an ADL icon is selected, the 3D model animates the action to show how the patient's bones would move to perform the selected ADL. The animation of full body skeleton is displayed on at least a portion of the user interface to illustrate the overall ADL motion. Under the skeleton animation, a progress bar shows the animation progress. If impingement occurs, the animation frames with impingement are indicated on the progress bar with a red line or other visual indication.

The software may include an indication of impingement areas. When an ADL icon is selected and the 3D model animates, the areas of the model in which the impingement is detected are highlighted in red or other visual indication of the location.

In one example, for Functional Ranges of a model, the model may detect only the first animation frame with impingement and display on the screen with either the maximum joint angle achieved before impingement (e.g., elevation) or the activity percentage achieved before impingement (such as 50% or 75%).

Using these devices and processes, personalized (via scapula orientation) and clinically relevant (via activities of daily living and Functional Ranges) impingement detection analysis can help identify osteophytes and drive implant selection and placement to maximize range of motion.

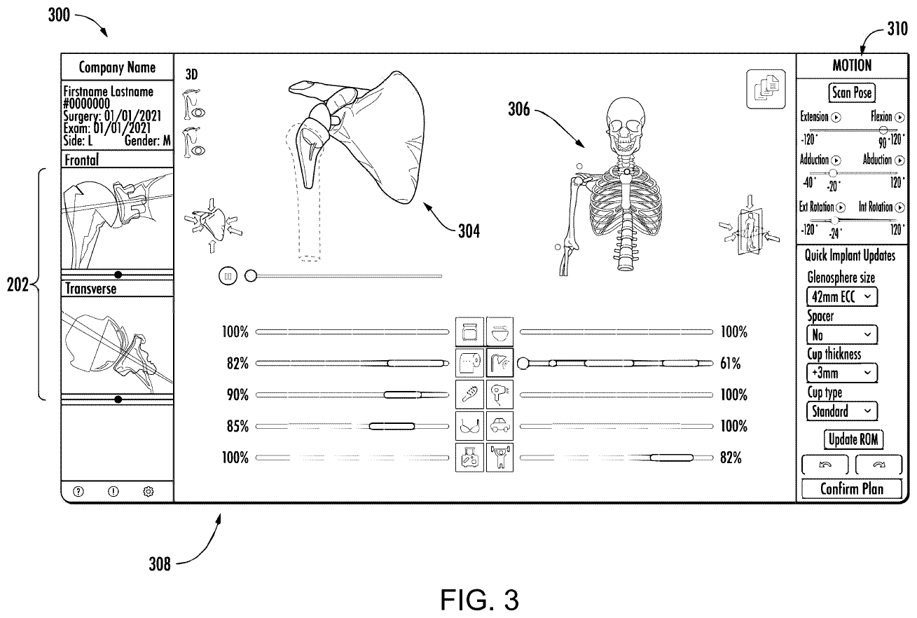

Activities simulation tool 112 provides a kinematic simulation of typical glenohumeral motion that occurs during activities of daily living. An analysis and visualization of the joint when moved within ranges-of-motion associated with various activities of daily living relevant to shoulder function, for example, reaching behind back, drinking from a cup, reaching the contralateral shoulder, etc. is provided in user interface screen 300 shown in FIG. 3. Sport-specific motions (e.g., a golf or tennis swing, etc.) can also be considered.

As with the user interface screen 200, exemplary user interface screen 300 shows one or more views 202 from the imagery used to generate the model of the patient's bone anatomy, model and placement of the implant shown thereon. The exemplary user interface screen shown in FIG. 3 also shows a reverse TSA procedure.

In some examples, generic shoulder kinematics are calculated from motion capture data of relevant activities and stored within the tool. In other examples, the kinematics can be derived from other sensors, for example, wearable sensors, fluoroscopy, x-ray, etc. Patient-specific shoulder kinematics can also be measured from the patient's contralateral joint, if healthy, and imported to the software to provide better patient-specific insight. Given an implant selection and placement, the user can select an activity of interest from a list of implemented activities 306. User interface screen 300 includes a model 304 similar to model 204 shown in FIG. 2. After the desired activity of daily living is selected from the list in 308, model 304 is animated to show the kinematic simulation during the selected activity of daily living. For example, the exemplary user interface screen 300 shows the showering activity highlighted within list 308. As such, the kinematic simulation of model 304 shows the ranges-of-motion required during a typical showering activity.

FIG. 3 shows a second method 306 of visualizing the ranges-of-motion. In this case, an animated skeleton 306 is shown with the shoulder joint animated to show the motions of the joint during the selected activity of daily living. As would be realized by one of skill in the art, the visualization 206 of the range-of-motion for the range-of-motion analysis and visualization tool shown in the user interface screen 200 of FIG. 2 may be used on this screen as well and, conversely, the visualization 306 for the activities analysis of visualization tool shown in FIG. 3. may be used as a visualization for the range-of-motion tool 110. Other methods of visualizing the range-of-motion are contemplated be within the scope of the present disclosure.

Potential bone-to-bone, implant-to-implant and/or implant-to-bone impingement is detected using the same collision detection algorithm and will be displayed within the 3D visualization of the patient's shoulder 308. In addition, the ranges-of-motion for each of the activities of daily living for each of the main axes is also shown in area 310 of user interface screen 300. In user interface screen 300, the controls in area 310 may be manipulated to manually show the position of the bones of the joint in model 304 or the range-of-motion in visualization 306. As with the user interface screen 200 of FIG. 2, the colored portions of the sliders indicate available ranges-of-motion, while the grayed-out areas of the sliders indicate areas of impingement. In other examples, other types of controls may be used and may show the ranges-of-motion in other ways and are contemplated to be within the scope of the present disclosure. The list of typical activities of daily living 308 also includes a visualization of the percentage of the activity cycle that cannot be performed without impingement, wherein the colored areas in the bars next to each icon for each activity of daily living show potential restrictions of the range-of-motion wherein the colors of the areas may be varied to show the severity of the restriction.

Suite 108 includes muscle analysis tool 114. As previously discussed, during the reverse TSA procedure, the center of rotation of the joint may change which may cause the moment arm and/or path of the muscles to change. In addition, the muscles may be required to lengthen or shorten depending upon the change in the path. In some examples, muscle analysis tool 114 provides muscle-driven simulations of the joint to provide an estimation of realistic implant contact mechanics, which can be used to calculate mechanical quantities related to risk of implant failure such as rotational moment at the implant-bone interface, implant micromotion, and implant wear. In other examples, the muscle analysis tool 114 could provide other analyses, for example, moment arm calculations throughout the joint range-of-motion or throughout activity cycle, force-generating capacity throughout the joint range-of-motion or throughout activity cycle, etc.

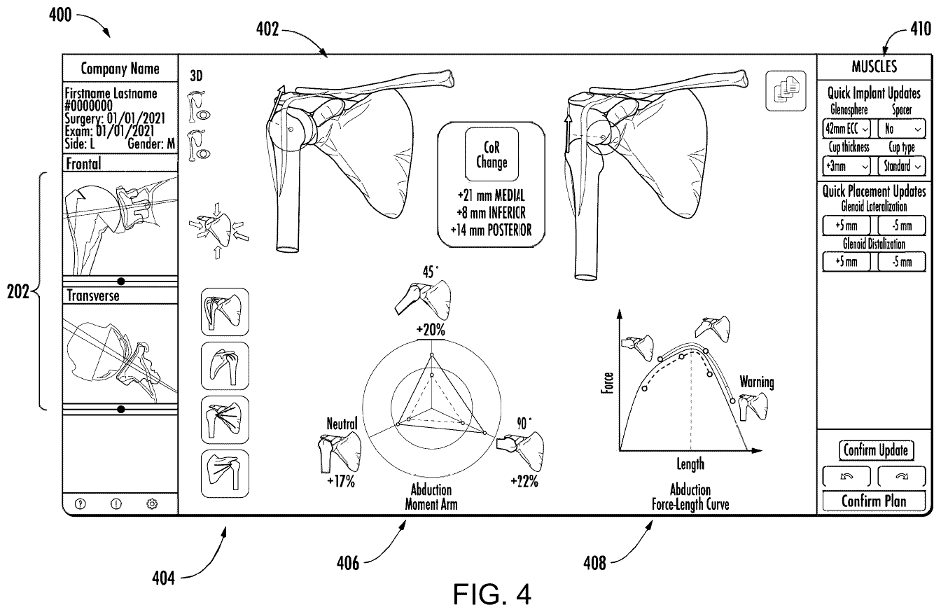

FIG. 4 shows exemplary user interface screen 400 for the muscle analysis tool 114. Model 402 shows various views of a selected muscle on the model and the change in the center of rotation of the joint from the native anatomy of the patient. The particular muscle displayed on model 402 may be selected using one of buttons 404 which, when selected, changes the muscle shown in model 402. A change in the center of rotation of the joint will also cause a change in the moment arm of various muscles of the joint (i.e., the distance between the muscle and the center of rotation of the joint), which is indicative of the ability of the muscle to generate a particular motion. The greater the moment arm of the muscle the easier it is for the muscle to manipulate the bones. Area 406 of user interface screen 400 visualizes the change in the moment arm, from the natural state to a predicted state post-surgery. User interface screen 400 also may provide a force-length curve 408 which provides a comparison of the length of the muscle in its natural state as opposed to the predicted post-operative length. A selection of any one of the buttons in list 404 will change each of model 402, showing the change in the center of rotation and display 406 showing the change in the moment arm and force-length curve 408 to reflect visualization of the muscle that has been selected using button 404. User interface screen 400 also includes area 410 which shows an exemplary interface for changing the positioning of the components of the implant.

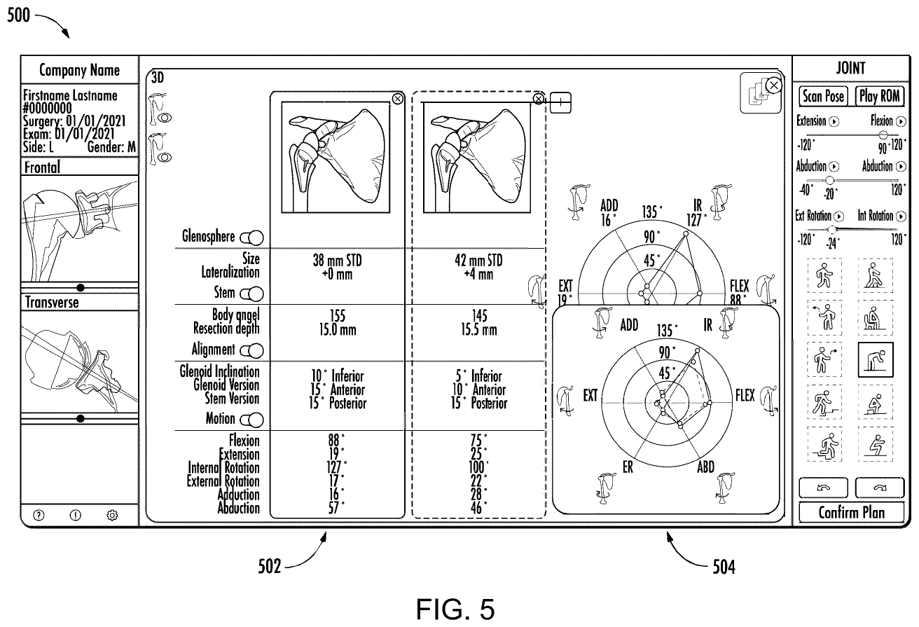

In various examples of the present disclosure, modeling tools 110, 112 and 114 in suite 108 can be accessed by the user at any time as long as an implant is fully selected and positioned. Alternatively, the user can store an implant selection at step 106 of workflow 100 within a list of comparison cases and the modeling tools will present an intuitive comparison of the calculated biomechanical quantities between selected cases. A user interface screen 500 for the comparison tool 116 is shown in FIG. 5. Comparison tool 116 allows a side-by-side comparison 502 of various characteristics of the joint resulting when implants and the positioning of their components is varied. Various aspects of the joint may also be shown. For example, a visualization 504 comparing the ranges-of-motion provided by either of the selected implants may be provided, with the ranges-of-motion for the different implant shown in different colors within visualization 504.

In various examples, any or all of tools 110, 112 and 114 may or may not be used by the user. For example, the muscle analysis tool 114 is most useful for reverse TSA procedures but may not useful when an anatomical TSA procedure undertaken.

In certain examples of the present disclosure, modeling and simulation results from a number of patient-specific cases may be used to generate artificial intelligence or machine learning based implant selection and placement clusters. Such clusters may be representative of the surgeons planning philosophies or techniques, which may be used to provide initial placement and selection within the planning tool that better align with the surgeon's preferences or the patient's preferences or with other post-operative measures. In certain other examples, the system may suggest implant positions that optimize certain parameters such as muscle moment arm, force-length curve, or minimal impingement.

The modeling tools 110, 112 and 114, the comparison tool 116 as well as the user interface allowing the user to select and specify the implant and the placement of the components of the implant may be implemented as one or more applications executing on a system comprising a processor and memory, storing software that, when executed by the processor, performs the functions of the one or more applications comprising the method.

The intra-operative workflow 200 may be implemented as software application running on a computing system interfaced with a navigated or robotic-assisted surgical platform or, alternatively, as part of the software of the navigated or robotic-assisted surgical platform.

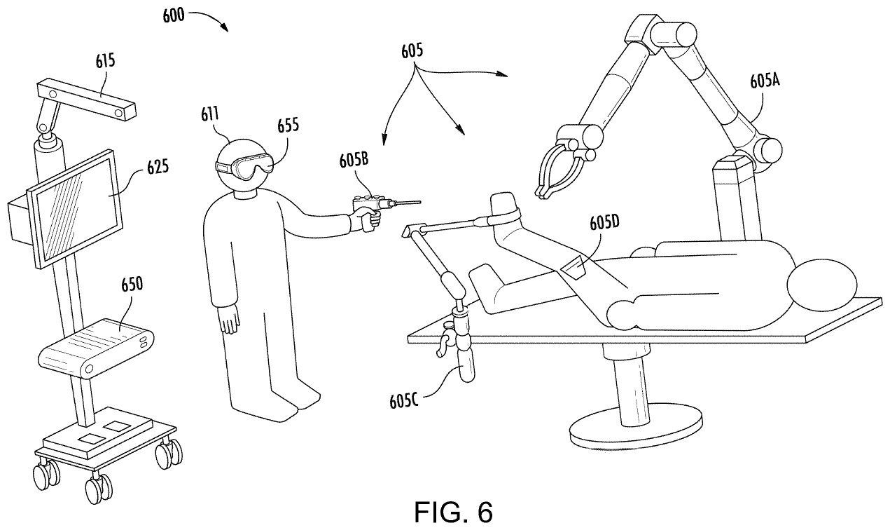

FIG. 6 provides an illustration of an exemplary computer-assisted surgical system 600, with which the system and method of the present disclosure may be implemented. System 600 uses computers, robotics, and imaging technology to aid surgeons in performing orthopedic surgery procedures such as total shoulder arthroplasty. For example, surgical navigation systems can aid surgeons in locating patient anatomical structures, guiding surgical instruments, and implanting medical devices with a high degree of accuracy. Surgical navigation systems 600 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 605 positions surgical tools relative to a patient during surgery. The exact components of the effector platform 605 will vary, depending on the example employed. For example, for a knee surgery, the effector platform 605 may include an end effector 605B that holds surgical tools or instruments during their use. The end effector 605B may be a handheld device or instrument used by the surgeon (e.g., a NAVIO® or CORI® hand piece or a cutting guide or jig) or, alternatively, the end effector 605B can include a device or instrument held or positioned by a robotic arm 605A.

The effector platform 605 can include a limb positioner 605C for positioning the patient's limbs during surgery. One example of a limb positioner 605C is the SMITH & NEPHEW SPIDER2 system. The limb positioner 605C may be operated manually by the surgeon or alternatively change limb positions based on instructions received from the surgical computer 650 (described below).

Resection equipment (e.g., end effector 605B) in FIG. 6) performs bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, and laser ablation systems. In some examples, the resection equipment is held and operated by the surgeon during surgery. In other examples, the effector platform 605 may be used to hold the resection equipment during use.

The effector platform 605 can also include a cutting guide or jig 605D that is used to guide saws or drills used to resect tissue during surgery. Such cutting guides 605D can be formed integrally as part of the effector platform 605 or robotic arm 605A or cutting guides can be separate structures that can be removably attached to the effector platform 605 or robotic arm 605A. The effector platform 605 or robotic arm 605A can be controlled by the system 600 to position a cutting guide or jig 605D adjacent to the patient's anatomy in accordance with a pre-operatively or intra-operatively developed surgical plan such that the cutting guide or jig will produce a precise bone cut in accordance with the surgical plan.

The tracking system 615 uses one or more sensors to collect real-time position data that locates the patient's anatomy and surgical instruments. For example, for TKA procedures, the tracking system 615 may provide a location and orientation of the end effector 605B during the procedure. In addition to positional data, data from the tracking system 615 can also be used to infer velocity/acceleration of anatomy/instrumentation, which can be used for tool control. In some examples, the tracking system 615 may use a tracker array attached to the end effector 605B to determine the location and orientation of the end effector 605B. The position of the end effector 605B may be inferred based on the position and orientation of the tracking system 615 and a known relationship in three-dimensional space between the tracking system 615 and the end effector 605B. Various types of tracking systems may be used in various examples of the present disclosure including, without limitation, Infrared (IR) tracking systems, electromagnetic (EM) tracking systems, video or image based tracking systems, and ultrasound registration and tracking systems.

The display 625 provides graphical user interfaces (GUIs) that display images and the user interface of the navigated surgical platform, as well as the user interface of the systems and methods of the present disclosure, shown in FIGS. 2-5, as well other information relevant to the surgery. For example, in some examples, the display 625 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 625 may include, for example, one or more computer monitors. As an alternative or supplement to the display 625, one or more members of the surgical staff may wear an augmented reality (AR) Head Mounted Device (HMD). For example, in FIG. 6 the surgeon 611 is wearing an AR HMD 655 that may, for example, overlay pre-operative image data on the patient or provide surgical planning suggestions. Various example uses of the AR HMD 655 in surgical procedures are detailed in the sections that follow.

Surgical computer 650 provides control instructions to various components of system 100, collects data from those components, and provides general processing for various data needed during surgery. In some examples, the surgical computer 650 is a general purpose computer. In other examples, the surgical computer 650 may be a parallel computing platform that uses multiple central processing units (CPUs) or graphics processing units (GPU) to perform processing. In some examples, the surgical computer 650 is connected to a remote server over one or more computer networks (e.g., the Internet). The remote server can be used, for example, for storage of data or execution of computationally intensive processing tasks.

In various examples, the one or more applications 110, 112, 114 and 116, as well as workflow 100 of the present disclosure may be implemented as one or more software applications executing on surgical computer 650 integrated with or separate from the software controlling the navigated surgical platform. Also, in various examples, the pre-operative workflow 100 may be implemented as a software application executing on a computing platform other than surgical computer 650, such as a laptop or tablet computing device. The computing device executing workflow 100 may be in communication with surgical computer 650 such as to provide the ability to transfer the results of the analyses to surgical computer 650.

Additional measurements and implant parameters may be used to increase the accuracy and effectiveness of the pre-surgery analysis of the implant parameters.

For example, a scapula coordinate system is defined using three landmarks: glenoid center, medial border, and inferior angle. A humerus coordinate system is defined using the head center landmark, the canal axis, and the neck axis.

In another example, a neutral position of joint is defined squaring the scapula and humerus coordinate systems to the global coordinate system.

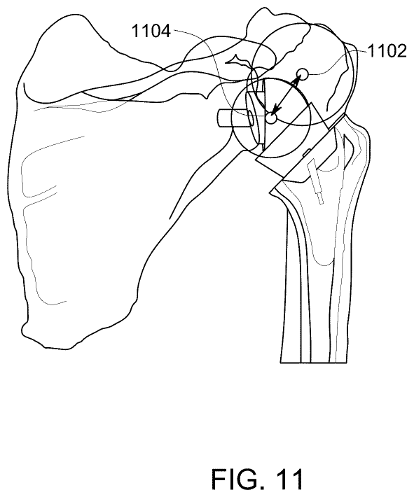

FIG. 10 illustrates an acromiohumeral distance and a lateral humeral offset.

Acromiohumeral distance (AHD) 1004 is measured as the vertical distance between the acromion most lateral point and the humerus greater tuberosity highest point when both the scapula and humerus are aligned to their neutral position.

Lateral humeral offset (LHO) 1002 is measured as the horizontal distance between the acromion most lateral point and the humerus most lateral point when both the scapula and humerus are aligned to their neutral position.

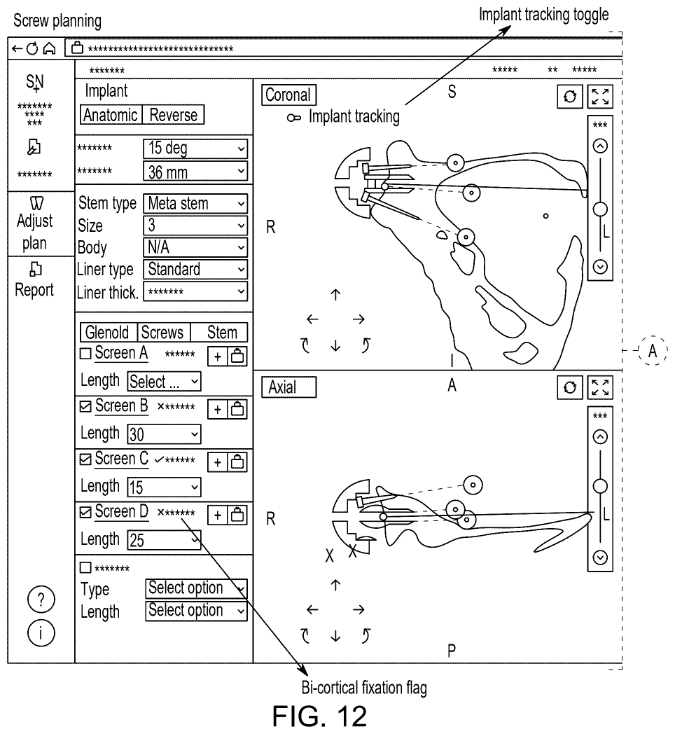

FIG. 11 illustrates a rotational center change.

Rotation center change is measured as the distance between the native humerus head center 1102 and the implant rotation center 1004. The implant rotation center 1104 is the glenosphere center or humeral head center. The distance is measured with both the scapula and humerus aligned to their neutral position, and it is divided into its three components (superior-inferior, medial-lateral, and anterior-posterior).

The DA-DGT distance is measured as the difference between the glenosphere center to acromion distance (DA) and the glenosphere center to greater tuberosity distance (DGT).

These measurements described herein, such as the AHD 1104, LHO 1102, rotational center change, and the DA-DGT distance are calculated in real time while the user modifies the pre-operative plan. These measurements are typically measured post-operatively in X-rays or using other technologies. However, in the technology described herein, the measurements are gathered and displayed in real time and updated on the model to inform decisions pre-operatively.

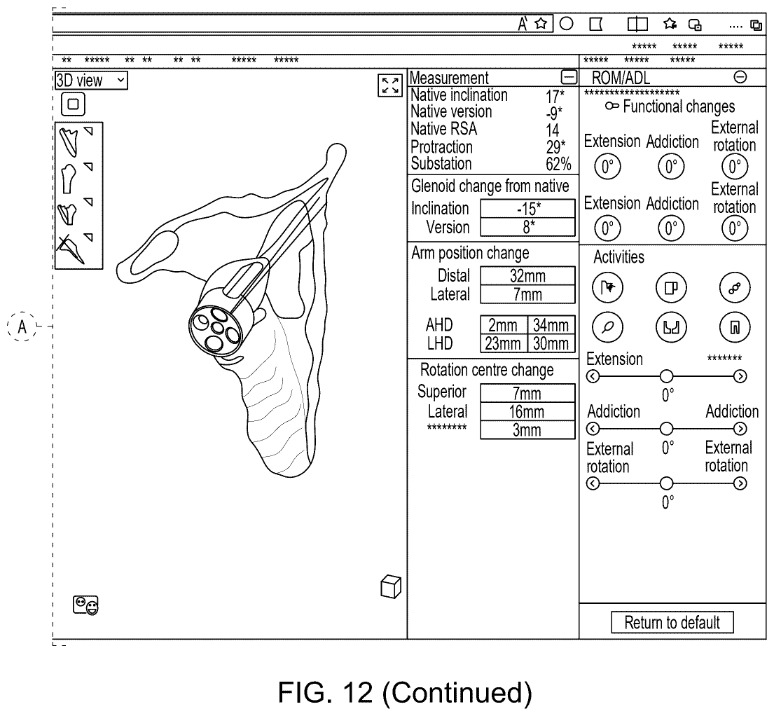

FIG. 12 illustrates a screw planning user interface for the system.

The system may include a screw pre-operative planning tool that includes screw visualization and orientation change controls on 2D cross sections and a bi-cortical fixation flag.

Screws trajectory, type, and length can be configured in a configured section of the user interface, such as a “Screws” tab. Each screw can be selected by actuating a visual icon, bubble, or other selection tool on the user interface. When selected, options for the selected screw or the other device can be presented to the user for selection. The user can configure different characteristics for the screw, such as the type of screw or the length of the screw. Screws can be added or removed by checking and unchecking an interface object, such as box next to an identifier for each screw, such as a screw name.

The system provides an option to display the screws and the bone sections in the 3D view or in one or more 2D cross-sections. By rotating the view and the screws in 2D cross-sections of the model, the user is able to more accurately select bone regions and screw lengths to achieve desired screw positions, such as to achieve a bi-cortical screw fixation.

In an example, screws can be added or removed from the implant model by checking and unchecking a selection object such as a bubble or a box on the user interface next to each screw's name or other identifier. In another example, a screw can be selected by clicking on the screw name in an interface panel of the operator interface.

After selecting a particular screw, a trajectory of that screw into the implant and the bone can be modified using the rotation arrows in the cross-section views once the screw is selected. In the example, the rotation of the screws and/or the bone section may be performed using the 2D cross-section. For example, the cross-sectional view may be rotated to achieve a desired angle for viewing the screw penetration. Alternatively, the screw is moved by directly dragging the screw user interface object within the cross-section view.

When toggling on an interface object such as “Implant Tracking” in the Coronal cross-section, the cross-section planes will follow the selected screw axis, making it easier for the user to always display a central cross-section of the screw while planning its trajectory.

In the user interface, the rotation of the screw in the 2D cross-section allows for a user to determine if bi-cortical fixation is achieved. If bi-cortical fixation is achieved, that indicates that the screw has entered one side of a bone, extends through the bone, and exits from an opposing side of the bone. The tip of the screw may extend a minimal distance through the opposing side of the bone. By extending through the opposing side of the bone, the screw is fixed in a second cortical section of the bone, which causes a more secure fixation of the screw.

In the user interface, a visual indication may signal that the screw has achieved bi-cortical fixation. For example, a flag or other signal may change color, shape, or size to indicate that bi-cortical fixation is achieved. In one example, a green flag may indicate a positive result, and a red flag may indicate a negative result. The user may rotate the screw in the image, change the screw depth or the screw size, or perform any other manipulation until a bi-cortical fixation is indicated on the user interface.

In one example, different screws with different diameters, lengths, screw patterns, or other features may be selected to be used in a particular screw hole in the interface representation of the device. For example, the user may select a screw hole and then select a screw to be used in that hole from a presented list of screw options. The user may rotate the screw on the 2D image a configured rotational cone. In an example, a 20-degree cone of rotation or any other suitable cone of rotation may be available to rotate the screw tip to achieve bi-cortical fixation.

This process provides easier control and visualization of rTSA peripheral screw position, produces more accurate plans, and saves time during surgery.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

The present disclosure has been described in the context of specific examples, which are intended only as examples of the disclosure. As would be realized, many variations of the described examples are possible. For example, variations in the design, shape, size, location, function and operation of various components, including both software and hardware components, would still be considered to be within the scope of the disclosure, which is defined by the following claims.

We claim:

1. A method of identifying impingement points in pre-operative planning of joint replacement surgery comprising:
deriving a model of the anatomy of a joint to be replaced from images gathered from one or more imaging modalities;
receiving a selection of an implant and placement of the implant on the anatomy of the joint;
analyzing and visualizing, on the model of the joint, a range-of-motion of the joint for the selected implant and placement of the implant; and
providing an animated visualization of the model through the range-of-motion,
wherein the animation applies scapulothoracic and glenohumeral angles to the model to simulate one or more of activities of daily living and functional ranges.

2. The method of claim 1, further comprising:
identifying, in the animation, one or more instances in which an impingement of the joint or the implant is indicated in the animation.

3. The method of claim 2, wherein the impingement is indicated via a visual interface object.

4. The method of claim 3, wherein the visual interface object changes a display based on a number of simulation frames that indicate impingements.

5. The method of claim 2, wherein the indicating of the impingement occurs in real time to provide data for selection of a placement of the implant.

6. The method of claim 1, wherein the animation of the ranges of motion comprises an animated skeletal representation of the joint.

7. A method for performing pre-operative planning of joint replacement surgery comprising:
deriving a model of the anatomy of a joint to be replaced from images gathered from one or more imaging modalities;
based on the model, calculating one or more of Acromiohumeral Distance (AHD), Lateral Humeral Offset (LHO), Rotation Center Change, and a difference between the glenosphere center to acromion distance (DA) and the glenosphere center to greater tuberosity distance (DGT); and
displaying the calculations in real time on a user interface while receiving a selection of an implant and placement of an implant on the anatomy of the joint.

8. The method of claim 7, wherein the calculations are based on a scapula coordinate system that is defined using three landmarks: a glenoid center, a medial border, and an inferior angle.

9. The method of claim 7, wherein the AHD is measured as a vertical distance between an acromion most lateral point and a humerus greater tuberosity highest point when both a scapula and humerus are aligned to a neutral position of the joint.

10. The method of claim 7, wherein the LHO is measured as a horizontal distance between an acromion most lateral point and a humerus most lateral point when both a scapula and the humerus are aligned to their neutral position.

11. The method of claim 7, wherein the rotation center change is measured as a distance between a native humerus head center and an implant rotation center when both a scapula and a humerus are aligned to neutral position.

12. The method of claim 11, wherein the rotation center change is divided into three components of superior-inferior, medial-lateral, and anterior-posterior.

13. The method of claim 9, wherein the neutral position is identified by squaring the scapula and humerus coordinate systems to a global coordinate system.

14. A method of planning screw placements during pre-operative planning of joint replacement surgery comprising:
deriving a three-dimensional model of the anatomy of a joint to be replaced from images gathered from one or more imaging modalities;
receiving a selection of an implant and placement of the implant on the anatomy of the joint;
selecting a two-dimensional cross section of the three-dimensional model; and
rotating a selected screw through the two-dimensional cross section to identify a placement.

15. The method of claim 14, wherein the identified placement indicates that the screw placement causes a bi-cortical fixation.

16. The method of claim 14, wherein the screw is rotatable along a configured cone of rotation.

17. The method of claim 14, wherein a user interface of the model provides an indication when a placement of the screw indicates a bi-cortical fixation.

18. The method of claim 14, wherein the screw is selectable via an interface object on a user interface of the model.
